(19) United States
(12) Patent Application Publication
(10) Pub. No.: US 2025/0187247 A1
(43) Pub. Date: Jun. 12, 2025

(54) Method for decontaminating the inside of preforms

(71) Applicant: SIDEL PARTICIPATIONS, OCTEVILLE-SUR-MER (FR)

(72) Inventor: Sandy LETELLIER, OCTEVILLE-SUR-MER (FR)

(21) Appl. No.: 18/972,850

(22) Filed: Dec. 7, 2024

Related U.S. Application Data
(60) Provisional application No. 63/609,095, filed on Dec. 12, 2023.

(30) Foreign Application Priority Data
Dec. 8, 2023 (FR) FR2313854

(51) Int. Cl. B29C 49/42; A61L 2/10; A61L 2/18; A61L 2/22; A61L 2/26; A61L 101/02; B29C 49/64

(52) U.S. Cl. CPC B29C 49/42418; A61L 2/10; A61L 2/186; A61L 2/22; A61L 2202/11; A61L 2202/15; A61L 2202/23; B29C 49/6418

(57) ABSTRACT

A Process for decontaminating an inner face of a neck of a preform made of thermoplastic material running along a production path in an installation for manufacturing containers. In example embodiments, the decontamination process includes, in succession, at least a treatment step and a sterilizing activation step. The treatment step includes exposing at least the inner face of the neck to a sterilizing jet having a sterilizing agent in a first treatment zone of the production path, and the sterilizing agent activation step includes directly exposing the inner face of the neck thus coated with sterilizing agent to ultraviolet radiation, in a second activation zone of the production path located downstream of the treatment zone.

### CROSS-REFERENCE TO RELATED APPLICATION

This application claims priority to U.S. Provisional Patent Application Ser. No. 63/609,095 filed on Dec. 12, 2023 and to French Patent Application No. FR2313854 filed on Dec. 8, 2023, the entireties of which are incorporated by reference herein for all purposes.

### TECHNICAL FIELD OF THE INVENTION

The invention relates to a method of decontaminating an inner face of a neck of a thermoplastic preform running along a production path in a container manufacturing plant.

### TECHNICAL BACKGROUND TO THE INVENTION

Containers made of thermoplastic material, such as polyethylene terephthalate (PET), are known to be produced by a process of stretch blow molding preforms.

In general, a preform has an axisymmetric shape. The preform has a neck that already has its final shape, while a body of the preform is intended to be deformed during the forming process. The main axis of the preform passes through the center of the neck.

To allow deformation, the body of the preform is heated above a glass transition temperature, making the wall of the body malleable by significantly reducing its elastic limit. On the other hand, the neck is kept at a temperature below the glass transition temperature to prevent deformation.

When the container is formed, a compressed forming fluid is injected into the body of the preform at a blowing pressure so as to “inflate” the body of the preform until it reaches its final shape.

Examples of a process for decontaminating a thermoplastic preform intended to be transformed into a container in a manufacturing plant are known from the state of the art.

The state of the art distinguishes, on the one hand, decontamination processes designed to decontaminate the inside of the preform and, on the other hand, decontamination processes designed to decontaminate the outside of the preform, in particular the neck of the preform.

Decontamination of the inside of the neck is a very important operation. Sufficient decontamination of this surface is a vital criterion, particularly for the manufacture of containers intended to contain food products. However, the inside of the neck is difficult to access.

Chemical decontamination processes are known, obtained by exposing the preform to a sterilizing agent such as hydrogen peroxide (H2O2) or peracetic acid. Such a chemical agent has a high oxidizing power, which enables some of the micro-organisms (viruses, germs, spores, etc.) exposed to it to be eliminated.

However, it has been found that such a chemical decontamination process is not sufficiently effective to decontaminate preforms sufficiently, particularly when they are intended for food or pharmaceutical use.

To improve the effectiveness of the sterilizing agent, it is known to increase its action considerably by heating it, in particular by exposing it to infrared radiation. This heating breaks down the sterilizing agent into even more oxidizing species, such as free radicals. The sterilizing agent is then said to have been “activated”.

However, the fact that the neck of the preform cannot be heated to activate the sterilizing agent is an obstacle for those skilled in the art.

As an alternative to chemical decontamination, decontamination by irradiation with ultraviolet radiation is also known.

The thermoplastic materials making up the preforms are generally opaque to ultraviolet radiation. Decontamination of the external face of the neck using ultraviolet radiation is limited due to its superficial nature, as the ultraviolet radiation does not penetrate deeply. It is therefore necessary to expose the surfaces to be decontaminated directly to ultraviolet radiation. This requires access to the surfaces to be treated. In the case of decontaminating the inside of the neck of preforms, this means in particular that it is not possible to decontaminate preforms when they are held by a mandrel inserted into their neck.

However, irradiating the face of the preform with ultraviolet radiation is less effective on certain types of micro-organisms such as molds than chemical decontamination using a sterilizing agent such as hydrogen peroxide (H2O2).

Generally, in most known solutions, ultraviolet treatment is opposed to chemical treatment, the two treatments being mutually exclusive.

It has already been proposed to activate the sterilizing agent using ultraviolet radiation instead of infrared radiation.

This is achieved by passing the preforms through a mist of hydrogen peroxide before exposing them to ultraviolet radiation. This achieves greater decontamination than ultraviolet irradiation alone.

However, this process is aimed at decontaminating preforms as a whole without specifically targeting the inside face of the neck, when the preforms pass through a feed device of a manufacturing installation.

The function of such a feeder is to straighten and align preforms that are delivered in bulk. After alignment and straightening, the preforms are generally received in an accumulation line between guide rails, with the preforms free to slide and come into contact with each other. The movement of the preforms is then not controlled and the preforms are free to swing between the rails, particularly under the effect of preforms colliding with each other.

However, the internal face of the neck of the preform is not uniformly covered by the sterilizing agent, the hydrogen peroxide mist being deposited on the said face in the form of multiple droplets between which however remain uncovered areas, particularly in the internal face of the neck which remains poorly accessible to passive exposure to the hydrogen peroxide mist.

In addition, in such a decontamination process, the mist tends to escape and settle on surrounding parts of the installation. As the sterilizing agent is extremely corrosive, it needs to be diluted significantly to avoid damaging the surrounding components exposed to it too quickly. This further reduces the effectiveness of this decontamination method.

In addition, exposing the preforms to ultraviolet radiation in such a supply device has a number of disadvantages. The duration of exposure of the preforms to ultraviolet radiation is not controlled and cannot be repeated.

In addition, it is necessary to expose the preforms to ultraviolet radiation for a very long time, which is not suitable for current production rates.

In fact, in such a feeder, there are variations in the speed at which the preforms run depending on the operating conditions of the downstream manufacturing installation. If the preforms are blocked or slowed down, they may be exposed to ultraviolet radiation for too long, which causes crystallization of the thermoplastic material, making them unsuitable for forming acceptable containers.

In addition, as the preforms are not held individually, some preforms may sway or be tilted as they pass through the ultraviolet radiation. As a result, an area of the internal faces of the neck may be under-exposed.

Generally speaking, there is a growing desire to increase the degree of decontamination, particularly in the packaging of food products. Decontamination must also be compatible with very high production rates, for example up to at least 60,000 preforms per hour.

New solutions are therefore being sought to improve the degree of decontamination obtained and the types of micro-organisms destroyed during decontamination operations on the outer face of a preform, in particular the inner face of the neck of the preform.

### BRIEF SUMMARY OF THE INVENTION

The invention proposes a process for decontaminating an internal face of a neck of a preform made of thermoplastic material running along a production path in a container manufacturing installation, each preform being held by an individual holding member, the said decontamination process comprising successively at least:
a treatment step consisting of exposing at least the internal face of the neck to a sterilizing jet comprising a sterilizing agent in a first treatment zone of the production path; and
a sterilizing agent activation step consisting of directly exposing the internal face of the neck thus covered with sterilizing agent to ultraviolet radiation, in a second activation zone of the production path located downstream of the treatment zone.

According to another characteristic of the process carried out according to the teachings of the invention, during the activation step, the preforms are constantly moving along the production path.

According to another characteristic of the process carried out according to the teachings of the invention, during the treatment step, the preforms are constantly moving along the production path.

According to another characteristic of the process carried out according to the teachings of the invention, during the treatment step, the sterilizing jet is directed exclusively towards the inside of the preform to be treated.

According to another feature of the process carried out in accordance with the teachings of the invention, the sterilizing jet is projected by a nozzle which moves along with the preform to be treated in the treatment zone.

According to another characteristic of the process carried out according to the teachings of the invention, the ultraviolet radiation is produced by at least one source which is arranged in a fixed manner with respect to the production path.

The invention also proposes an installation for manufacturing containers from preforms made of thermoplastic material, implementing the process according to the teachings of the invention, the installation comprising at least one device for conveying the preforms in line along a production path, the conveying means comprising members for individually holding the preforms as they move, the installation comprising:
a preform treatment zone through which the production path passes, and which includes at least one nozzle for spraying a sterilizing jet including a sterilizing agent intended to be directed directly towards the inside face of the neck of the preforms passing through the treatment zone;
a zone for activating the sterilizing agent, which comprises at least one source of ultraviolet radiation intended to be directed directly towards the inner face of the neck of the preforms passing through the activation zone, the activation zone being located downstream of the treatment zone.

According to another characteristic of the installation produced in accordance with the teachings of the invention, the installation comprises a first device for conveying into the treatment zone comprising associated holding members moving along a closed circuit, each holding member being associated with a nozzle for projecting the said sterilizing jet moving jointly with the holding member along the closed circuit.

According to another characteristic of the installation produced in accordance with the teachings of the invention, the installation comprises a second device for conveying into the activation zone comprising associated holding members moving along a closed circuit, the at least one source of ultraviolet radiation being arranged in a fixed manner in the activation zone so that the holding members move past the said sources.

According to another characteristic of the installation carried out in accordance with the teachings of the invention, the installation comprises a unit for thermal conditioning of the preforms, the activation zone being arranged upstream of the thermal conditioning unit.

According to another characteristic of the installation carried out in accordance with the teachings of the invention, the installation comprises a unit for thermal conditioning of the preforms, the activation zone being arranged downstream of the thermal conditioning unit.

According to another characteristic of the installation produced according to the teachings of the invention, the first conveying device is formed by a wheel on the periphery of which the holding members are arranged.

According to another characteristic of the installation, the second conveying device is formed by a wheel on the periphery of which the holding members are arranged.

According to a further characteristic of the installation carried out in accordance with the teachings of the invention, the retaining members are formed by notches.

According to some example characteristics, the term process and the term method are used interchangeably to refer to the systematic approach for decontaminating the inner face of a neck of a thermoplastic preform. According to other example characteristics, “process” encompasses the entire sequence of actions taken to achieve decontamination, and “method” refers to the specific techniques or steps within that process.

### BRIEF DESCRIPTION OF THE DRAWINGS

Other features and advantages of the invention will become apparent in the course of the detailed description which follows, for the understanding of which reference is made to the appended drawings briefly described below.

FIG. 1 is a top view showing schematically a container manufacturing plant constructed in accordance with the teachings of the invention.

FIG. 2 is a side view showing a preform intended to be decontaminated according to the process of the invention in the plant shown in FIG. 1.

FIG. 3 is a top view showing details of a first treatment zone in the installation shown in FIG. 1.

FIG. 4 is a cross-sectional view along sectional plane 4 4 of FIG. 3, showing a preform undergoing a first processing step in the processing zone of the installation.

FIG. 5 is a top view showing in detail a second activation zone of the installation in FIG. 1.

FIG. 6 is a sectional view along sectional plane 6 6 of FIG. 5, showing a preform undergoing a second activation step in the activation zone of the installation.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

In the remainder of the description, elements having an identical structure or similar functions will be referred to by the same references.

In the remainder of the description, the terms “upstream” and “downstream” will be used to refer to the direction of movement of the preforms along their production path.

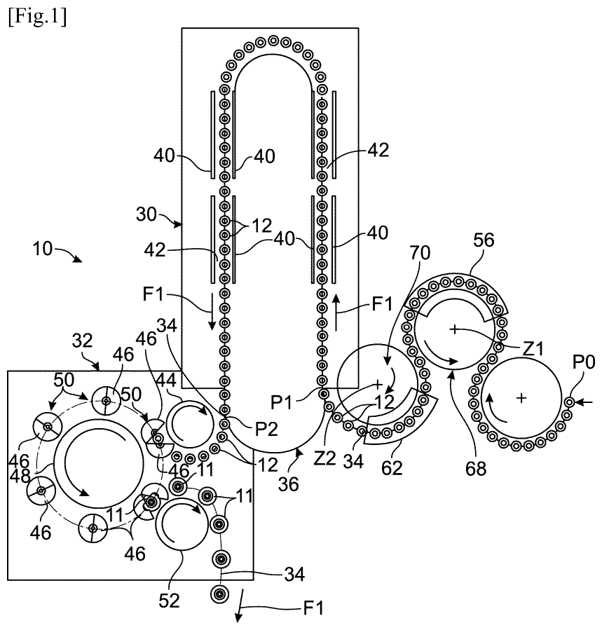

FIG. 1 schematically illustrates a plant 10 for manufacturing containers 11 from preforms 12 made of thermoplastic material and more particularly PET (polyethylene terephthalate).

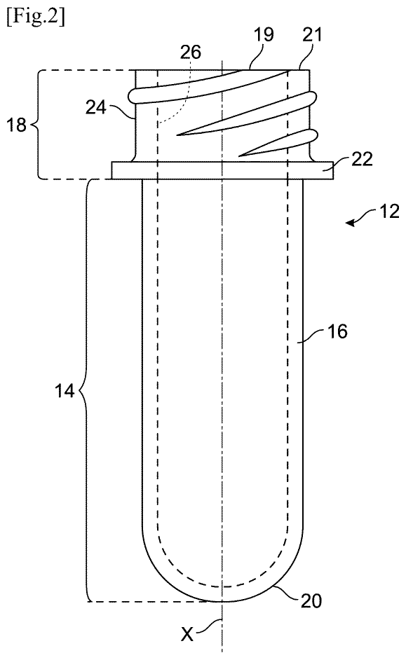

As shown in FIG. 2, each preform 12 comprises a cylindrical body 14 with an “X” axis. The body 14 has a side wall 16 which defines an internal volume. The wall 16 has a shape of revolution about the axis “X”.

An upper end of the body 14 opens into a neck 18 with an opening 19 delimited radially by a lip 21. The neck 18 has the final shape of that of the container 11 to be obtained. As a result, the neck 18 must not undergo the slightest deformation during manufacture of the container 11. The body 14 has a base 20 which closes its lower end and is generally hemispherical in shape. The neck 18 has a collar 22 at its junction with the body 14. The lower face of the collar 22 is intended to form a support face to enable the preform 12 to be supported during molding and/or transport.

The neck 18 is delimited by an external face 24 and an internal face 26. The internal face 26 has a cylindrical shape with an “X” axis.

At the end of injection molding, the preforms 12 are cooled to give the thermoplastic material an amorphous state. It is thus possible to make the thermoplastic material malleable again by heating above a glass transition temperature.

Referring again to FIG. 1, the manufacturing installation 10 comprises a thermal conditioning unit 30 and a forming unit 32.

The preforms 12 move in line along a production path 34 that passes through the thermal conditioning unit 30 and the forming unit 32. The direction of movement of the preforms 12 is indicated by the arrows “F1” in FIG. 1. During normal operation of the manufacturing plant 10, the preforms 12 are constantly moving along the production path 34.

The manufacturing installation 10 comprises means for conveying the preforms 12 in line along the production path 34. The conveying means comprise members 58, 66 for individually holding the preforms 12 as they move, which will be described in more detail later. In this way, the preforms 12 are constantly held individually throughout the production path 34.

The manufacturing installation 10 is generally supplied with preforms 12 by a device (not shown) for supplying preforms straightened and aligned in a row.

The production path 34 starts from the moment when the preforms 12, thus straightened and aligned in a row, are held individually by the individual holding members of the conveying means of the manufacturing installation 10. The start of the production path 34 is indicated here by the point PO.

The function of the thermal conditioning unit 30 is to heat the body 14 of the preforms 12 to a temperature greater than or equal to the glass transition of the constituent material, for example greater than 70° C. when this material is PET. The thermal conditioning unit 30 comprises a conveyor 36 (illustrated schematically) for transporting the preforms 12 by rotating them on themselves.

The conveyor 36 generally comprises mandrels (not shown) which are nested axially in the neck 18 to transport the preforms 12. The mandrel closes the opening 19 in the neck 18. This means that the inside face 26 of the neck 18 is completely inaccessible when the preforms 12 are circulating in the thermal conditioning unit 30. The mandrels move along a closed circuit. The mandrels are carried, for example, by the links of a chain or by independent shuttles moving along a rail.

The thermal conditioning unit 30 also includes heating means 40 for heating the preforms 12, for example lamps facing reflectors or laser sources that emit electromagnetic radiation for heating, in this case infrared radiation in the near infrared range.

The preforms 12 enter the thermal conditioning unit 30 as they are individually picked up by a mandrel of the conveyor 36, here at an entry point indicated by point P1. The conveyor 36 transports them along a U-shaped section of their production path 34 passing through a heating zone 42. The body 14 of the preforms is heated as it passes by the heating means 40, which may be positioned on one side or on either side of the preforms 12 in relation to their direction of travel.

On the other hand, the neck 18 of the preforms 12 is maintained at a temperature well below the glass transition temperature. To this end, the necks 18 are protected from infrared radiation.

The hot preforms 12 are extracted from the thermal conditioning unit 30 after passing through the heating zone 42 at an exit point P2. They are transferred to molds in the forming unit 32 by a transfer device 44, such as a transfer wheel, interposed between the thermal conditioning unit 30 and the forming unit 32.

The transfer wheel comprises arms (not shown, as they are known per se) which successively grip the preforms 12, as they leave the thermal conditioning unit 30, at their necks 18, to introduce each of them in turn into a mold 46 of the forming unit 32. The forming unit 32 comprises a rotating carousel 48 at the periphery of which are arranged a number of blowing stations 50 comprising a mold 46.

Each hot preform 12 leaving the thermal conditioning unit 30 is introduced into a mold 46 of the blowing station 50 to be blown and transformed into a container 11 by deformation of its body 14. Once completed, the container 11 is extracted from the blowing station 50 by a second transfer device 52.

It is important that the inner face 26 of the neck 18 of the preforms 12 is sufficiently decontaminated before the preform 12 reaches the blowing unit 32.

To this end, the invention proposes a process for decontaminating the inner face 26 of the neck 18 which is particularly effective, even at very high production rates, greater than or equal to 66,000 containers per hour.

The decontamination process is applied to the preforms 12 as they pass along the production path 34. It is important that each preform 12 is held by an individual holding member 58, 66 for the process to be effective.

The decontamination process successively comprises at least the following two steps.

A first treatment step “E1” consists of exposing at least the inner face 26 of the neck 18 to a sterilizing jet 54 containing a sterilizing agent in a first treatment zone 56 of the production path 34.

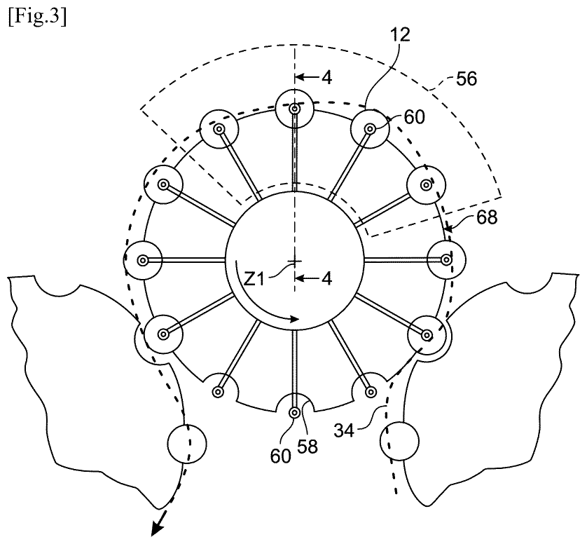

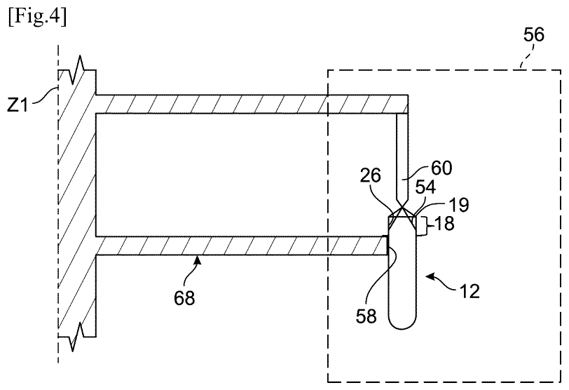

An example of this treatment step “E1” is shown in FIGS. 3 and 4.

The treatment zone 56 is arranged upstream of the thermal conditioning unit 30.

The sterilizing agent is hydrogen peroxide (H2O2), for example.

The sterilizing agent can be diluted in a diluent, such as water. For example, a solution of sterilizing agent diluted to at least 10%, for example diluted to 20% or 25%.

The sterilizing jet 54 can be formed by mixing the vaporized solution with a pressurized gas, known as the “carrier gas”, to enable the sterilizing jet 54 to be propelled. The carrier gas is, for example, compressed air.

This first treatment step “E1” is carried out by projecting at least one sterilizing jet 54 directed towards the inside of the preform 12 through the opening 19 while it is held by an individual holding member 58. More particularly, the sterilizing jet 54 is projected directly towards the inner face 26 of the neck 18 of the preform 12.

The sterilizing jet 54 is shaped to enable the internal face 26 of the neck 18 to be completely and uniformly covered.

Each sterilizing jet 54 is emitted axially by a nozzle 60 arranged axially in line with the opening 19 in the preform 12, above the neck 18. Advantageously, this enables the sterilizing agent to be deposited very precisely on the whole of the inner face 26 of the neck 18 of the preforms 12 in a uniform layer because the preforms 12 are held in position relative to the nozzle 60.

During the “E1” treatment step, the preforms 12 are constantly moving in the treatment zone 56.

Preferably, each nozzle 60 is movably mounted together with an associated holding member 58. In this way, the sterilizing jet 54 is projected by the nozzle 60 which moves together with the preform 12 to be treated in the treatment zone 56. Thus, during treatment step “E1”, the sterilizing jet 54 is directed exclusively towards the inside of the preform 12 to be treated, passing through the opening 19.

Preferably, during treatment step “E1”, the sterilizing jet 54 is directed exclusively towards the inside of the preform 12 to be treated.

As a result, it is possible to target exclusively the inner face 26 of the neck 18 of the preform 12 with the sterilizing jet 54 as it moves along the treatment zone 56. In particular, this makes it possible to use only the quantity of sterilizing agent required to treat the inner face 26 of the neck 18 without loss.

In a variant of the invention that is not shown, the treatment zone comprises a nozzle rail that is immobile relative to the floor so that each preform passes successively under each nozzle of the rail as it moves along the treatment zone. In this variant, the external face of the neck is also exposed to the sterilizing jets.

The process includes a second sterilizing agent activation step “E2” which takes place after the end of the first treatment step “E1”. This second activation step “E2” consists in directly exposing the inner face 26 of the neck 18 thus covered with sterilizing agent to ultraviolet (UV) radiation 61, in a second activation zone 62 located downstream of the treatment zone 56.

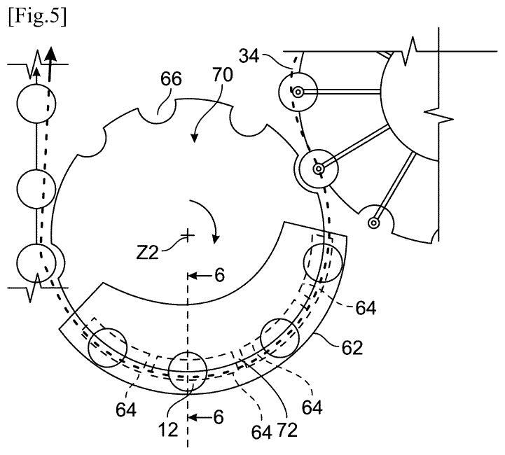

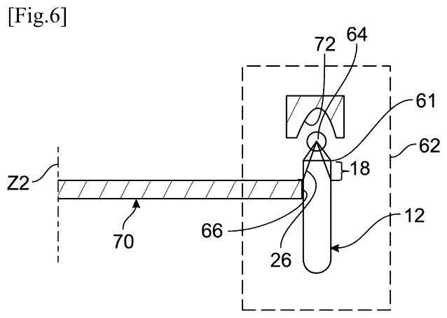

An example of this second activation step “E2” is shown in FIGS. 5 and 6.

The term “directly” means that the 61 UV radiation reaches the inner face 26 locally without passing through a wall of the preform 12. As explained in the preamble, preforms 12 are generally made of a material that does not allow UV radiation to pass easily.

The activation zone 62 is located upstream of the thermal conditioning unit 30. Alternatively, it can be arranged downstream of the thermal conditioning unit 30.

This is ultraviolet radiation 61 of the UVc type, for example, with a wavelength of between 100 nm and 280 nm. Preferably, the spectrum of ultraviolet radiation 61 has an emission peak of around 254 nm.

The power of the ultraviolet radiation 61 produced is, for example, of the order of 30 mW/cm².

The ultraviolet radiation 61 is produced by a source such as an amalgam lamp or a light-emitting diode (LED).

During the “E2” activation stage, the preforms 12 are constantly moving along the production path.

The ultraviolet radiation 61 is produced here by at least one source 64 which is arranged in a fixed manner with respect to the production path 34.

The preforms 12 are held individually by a holding member 66 as they pass through the activation zone 62. This promotes total exposure of the inner face 26 of the neck 18, in a repeatable and controlled manner for all the preforms 12.

The activation zone 62 comprises several sources 64 of ultraviolet radiation which are immobile relative to the ground so that each preform 12 passes successively under each source 64 as it moves along the activation zone 62, so that the UV radiation 61 penetrates the preform 12 through its opening 19 before directly reaching the inner face 26 of the neck 18.

In a non-represented variant of the invention, each source is mounted so as to be movable together with an associated holding member 66. In this way, the ultraviolet radiation is produced by a source which moves with the preform to be activated in the activation zone.

The fact that the preforms 12 are held by an individual holding member 66 as they pass through the activation zone 62 means that the entire inner face 26 of the necks 18 can be exposed for a controlled period of time.

By exposing the sterilizing agent covering the inner face 26 of the neck 18 to UV radiation, the sterilizing agent is broken down into even more oxidizing species, such as free radicals. The sterilizing agent is then said to have been “activated”.

Such a process carried out in accordance with the teachings of the invention makes it possible to obtain highly effective decontamination which makes it possible to divide by at least 100,000 the quantity of microorganisms present on the inner face 26 of the neck 18 just before the application of the decontamination process. In other words, the process according to the teachings of the invention makes it possible to obtain a logarithmic reduction of the contaminants present on the internal face 26 of the neck 18 of at least 5-log. This process is remarkable in that such a level of decontamination is obtained with exposure of the inner face 26 of the collars 18 to ultraviolet radiation with a duration of much less than a second, for example between 500 milliseconds and 3 seconds. This makes the process particularly suitable for very high container production rates, for example greater than or equal to 66,000 containers per hour.

To implement this process, the installation 10 for manufacturing containers 11 from preforms 12 made of thermoplastic material comprises means for conveying the preforms 12 in a row along the production path 34.

The plant 10 also comprises a zone 56 for treating the preforms 12, through which the production path 34 passes and which comprises at least one nozzle 60 for projecting a stream of sterilizing agent in the form of a sterilizing jet 54 containing the sterilizing agent intended to be directed directly towards the inner face 26 of the neck 18 of the preforms 12 passing through the treatment zone 56.

Installation 10 also includes a sterilizing agent activation zone 62, which comprises at least one source 64 of ultraviolet radiation 61 designed to be directed directly towards the inner face 26 of the neck 18 of the preforms 12 passing through the activation zone 62.

Activation zone 62 is located downstream of processing zone 56. The activation zone 62 is separate from the processing zone 56, so that the sterilizing agent is not exposed to ultraviolet radiation 61 until it has been deposited on the inner face 26 of the neck 18 of the preforms 12.

The conveying means comprise members 58, 66 for individually holding preforms 12 as they move. At least some of these conveying means comprise individual holding members 58, 66 which grip the preforms 12 from the outside. This leaves the opening 19 free, so that the inner face 26 of the collars 18 remains accessible from the outside, to enable them to be successively exposed to the sterilizing jet 54 and then to the ultraviolet radiation 61.

The mandrels described above are not suitable for gripping the preforms 12 from the inside, as they are inserted into the collars 18. This is why the treatment and activation zones 56, 62 are arranged upstream or downstream of the thermal conditioning unit 30. However, if the preforms were gripped in the heat-conditioning unit by holding means making the inside of their necks accessible, the treatment and activation zones could be located in the heat-conditioning unit.

Such conveying means comprise a first conveying device 68 in the treatment zone 56. This first conveying device 68 comprises associated holding members 58 moving along a closed circuit.

Each holding member 58 is associated here with a nozzle 60 for projecting said sterilizing jet 54, moving in conjunction with the holding member 58 along the closed circuit. The nozzles 60 are controlled so as to project the sterilizing jet 54 when they are located in the treatment zone 56.

Advantageously, the sterilizing jet 54 is stopped outside the treatment zone 56. This avoids spreading the sterilizing agent outside the preforms 12. In this way, the sterilizing agent is advantageously saved. In addition, since sterilizing agent is generally highly corrosive, this avoids creating a corrosive atmosphere likely to damage certain components of the 10 system.

The first conveying device (68) consists of a wheel on the periphery of which the holding devices (58) are arranged. This is, for example, a transfer wheel mounted so as to rotate relative to the ground around a vertical “Z1” axis. The closed circuit is thus circular in shape.

In a non-represented variant of the invention, the holding devices are carried by independent shuttles moving along a rail.

For example, the retaining members 58 are formed by notches on the periphery of which the underside of the flange 22 is pressed, as shown in FIG. 4.

Alternatively, but not pictorially, the holding devices can be formed by grippers which grip the preforms from the outside, below and/or above the flange.

This first conveying device (68) is located upstream of the thermal conditioning unit (30) and the entry point (P0) into the plant (10).

In the embodiment shown in the figures, the means for conveying preforms 12 into the activation zone 62 comprise a second conveying device 70 with associated holding members 66 moving along a closed circuit. The second conveying device 70 is separate from the first conveying device 68.

In an unrepresented variant of the invention, the holding members are carried by independent shuttles moving along a rail.

In an unrepresented variant of the invention, the activation zone and the treatment zone are arranged on the same conveying device.

The second conveyor device (70) consists of a wheel on whose periphery the holding devices (66) are arranged. This is, for example, a transfer wheel mounted so as to rotate relative to the ground around a vertical “Z2” axis. The closed circuit is thus circular in shape.

The retaining elements 66 are formed, for example, by notches on the periphery of which the underside of the flange 22 is pressed.

Alternatively, not shown here, the holding devices can be formed by grippers which grip the preforms from the outside, below and/or above the flange.

This second conveying device (70) is arranged upstream of the thermal conditioning unit (30) and downstream of the first conveying device (68).

In a non-representative variant of the invention, the second conveying device is arranged downstream of the thermal conditioning unit. Preferably, the second conveying device is arranged upstream of the forming unit.

In the embodiment shown in FIGS. 5 and 6, the activation zone 62 comprises ultraviolet radiation sources 64 that are fixed relative to the ground. As a result, the holding members 66 of the second conveying device 70 move past the said sources 64 when they are in the activation zone 62. In this way, the preforms 12 pass by, and with neck 18 directed towards the said sources 64 when they pass axially in line with the latter. In this way, the 61 UV radiation penetrates axially inside the preforms 12 through opening 19.

The sources 64 are, for example, amalgam lamps associated with reflectors 72 which direct the ultraviolet radiation 61 directly towards the inner face 26 of the neck 18 of the preforms as it enters through the opening 19.

The invention is applied here specifically to the decontamination of the inner face of the neck. However, the inner side of the body can benefit in the same way as the inner side of the neck, particularly if part of the sterilizing jet also exposes the inner side of the body and the ultraviolet radiation reaches the entire inner side of the body.

The decontamination process described in this manual thus enables preforms to be decontaminated very effectively, despite very high production rates. The process also protects the integrity of the neck 18 of the preforms 12 by not exposing them to infrared radiation.

Very advantageously, the fact that the preforms are held individually during each stage of the process means that the time of exposure to the sterilizing jet and the time of exposure to ultraviolet radiation of the inner face of the body can be very precisely controlled. In this way, the quantity of sterilizing agent applied to each preform, and the radiation dose for each preform, can be precisely controlled.

According to example embodiments, the present invention significantly improves internal neck decontamination performances, reduces reliance on the heating profile to achieve target decontamination performances, and reduces impacts of resins and additives on decontamination performances. In example embodiments, advantages of the present invention can include 1) good integration into the aseptic scheduled process; 2) the preforms are already containing hydrogen peroxide when exposed to ultraviolet radiation; and 3) is compact and capable of being retrofitted with existing manufacturing installations. Furthermore, according to example embodiments, one or more sensors can be provided for emissivity control of the ultraviolet light.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that measurements, amounts, and other numerical data can be expressed herein in a range format. It is also understood that there are a number of values disclosed herein, and that each value is also herein disclosed as “approximately” that particular value in addition to the value itself. For example, if the value “10” is disclosed, then “approximately 10” is also disclosed. Similarly, when values are expressed as approximations, by use of the antecedent “approximately,” it will be understood that the particular value forms a further aspect. For example, if the value “approximately 10” is disclosed, then “10” is also disclosed.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

1. A method of decontaminating an inside face of a neck of a preform of thermoplastic material passing along a production path in an installation for manufacturing containers, each preform being held by an individual holding member, said decontamination method comprising in succession at least:
a treatment step consisting in exposing at least the inner face of the neck to a sterilizing jet comprising a sterilizing agent in a first treatment zone of the production path; and
a sterilizing agent activation step consisting of directly exposing the inner face of the neck thus coated with sterilizing agent to ultraviolet radiation, in a second activation zone of the production path located downstream of the treatment zone.

2. The method of claim 1, wherein during the activation step, the preforms move constantly along the production path.

3. The method of claim 1, wherein during the treatment step, the preforms move constantly along the production path.

4. The method of claim 1, wherein during the treatment step, the sterilizing jet is directed exclusively towards the inside of the preform to be treated.

5. The method of claim 4, wherein the sterilizing jet is projected by a nozzle which moves together with the preform to be treated in the treatment zone.

6. The method of claim 1, wherein the ultraviolet radiation is produced by at least one source which is arranged in a fixed manner relative to the production path.

7. An installation for manufacturing containers from preforms made of thermoplastic material, implementing the method according to claim 1, the installation comprising at least one device for conveying preforms in a row along a production path, the conveying means comprising members for individually holding preforms during their displacement, the installation comprising:
a zone for treating the preforms, through which the production path passes and which comprises at least one nozzle for projecting a sterilizing jet comprising a sterilizing agent intended to be directed directly towards the inner face of the neck of the preforms passing through the treatment zone; and
a sterilizing agent activation zone comprising at least one source of ultraviolet radiation intended to be directed directly towards the inner face of the neck of the preforms passing through the activation zone, the activation zone being located downstream of the treatment zone.

8. The installation of claim 7, further comprising a first conveying device in the treatment zone comprising associated holding members moving along a closed circuit, each holding member being associated with a nozzle for projecting said sterilizing jet moving jointly with the holding member along the closed circuit.

9. The installation of claim 7, further comprising a second conveying device in the activation zone comprising associated holding members moving along a closed circuit, the at least one source of ultraviolet radiation being arranged in a fixed manner in the activation zone so that the holding members run past the said sources.

10. The installation of claim 7, further comprising a unit for thermal conditioning of the preforms, the activation zone being arranged upstream of the thermal conditioning unit.

11. The installation of claim 7, further comprising a unit for thermal conditioning of the preforms, the activation zone being arranged downstream of the thermal conditioning unit.

12. The installation of claim 8, wherein the first conveying device is formed by a wheel at the periphery of which the holding members are arranged.

13. The installation of claim 9, wherein the second conveying device is formed by a wheel on the periphery of which the holding members are arranged.

14. The installation of claim 12, wherein the holding members are formed by notches.

15. A method of decontaminating an inside face of a neck of a preform of thermoplastic material passing along a production path in an installation for manufacturing containers, the method comprising:
a treatment step consisting in exposing at least the inner face of the neck to a sterilizing jet comprising a sterilizing agent in a first treatment zone of the production path; and
a sterilizing agent activation step consisting of directly exposing the inner face of the neck thus coated with sterilizing agent to ultraviolet radiation, in a second activation zone of the production path located downstream of the treatment zone.

16. The method of claim 15, wherein during the activation step, the preforms move constantly along the production path.

17. The method of claim 1, wherein during the treatment step, the preforms move constantly along the production path.

18. The method of claim 15, wherein during the treatment step, the sterilizing jet is directed exclusively towards the inside of the preform to be treated.

19. The method of claim 18, wherein the sterilizing jet is projected by a nozzle which moves together with the preform to be treated in the treatment zone.

20. The method of claim 19, wherein the ultraviolet radiation is produced by at least one source which is arranged in a fixed manner relative to the production path.
